(19) United States
(12) Patent Application Publication
(10) Pub. No.: US 2007/0264219 A1
(43) Pub. Date: Nov. 15, 2007

(54) Cosmetic method for soothing the reactions of discomfort of the scalp, using an oxyalkylenated sorbitan ester as a soothing agent

(76) Inventors: Sandrine Decoster, Saint Gratien (FR); Patricia Mezure, Bougival (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/642,760

(22) Filed: Dec. 21, 2006

Related U.S. Application Data
(60) Provisional application No. 60/757,849, filed on Jan. 11, 2006.

(30) Foreign Application Priority Data
Dec. 22, 2005 (FR) 05 13192

(51) Int. Cl. A61K 8/92; A61Q 5/02; A61Q 5/12

(52) U.S. Cl. 424/70.11

(57) ABSTRACT

The present disclosure relates to a cosmetic method for soothing the reactions of discomfort of the scalp, wherein it comprises applying on the scalp a cosmetic composition comprising, in an aqueous or aqueous-alcoholic medium, at least one ester of fatty acid and sorbitan comprising from 1 to 20 oxyalkylene units as a soothing agent.

This application claims benefit of U.S. Provisional Application No. 60/757,849 filed Jan. 11, 2006, the contents of which are incorporated herein by reference. This application also claims benefit of priority under 35 U.S.C. § 119 to French Patent Application No. FR 05 13192, filed Dec. 22, 2005, the contents of which are also incorporated herein by reference.

The present disclosure relates to the use of a particular compound of the oxyalkylenated sorbitan ester family for soothing the scalp.

Additionally, the present disclosure also relates to a method of cosmetic treatment for soothing the scalp employing such an oxyalkylenated sorbitan ester.

Many people have a scalp that is naturally sensitive, giving rise to various effects of irritation and itching. Moreover, even a scalp that is not very sensitive may be irritated by external aggressive factors such as bad weather, sweat, frequent washing of the hair, and the use of aggressive cosmetic hair products, for example hair coloring or perming products, any of which may cause itching on one or more areas of the scalp.

Application of compositions, generally aqueous-alcoholic, on the scalp without rinsing is known as a remedy for this discomfort.

Moreover, the use of oxyalkylenated sorbitan esters as surfactants in compositions for cosmetic treatment of the hair has been described in the prior art.

Thus, French Patent Application No. 2 804 020 describes mild washing compositions, notably shampoos, comprising at least one detergent surfactant and at least one oxyethylenated ester of fatty acid and sorbitan having a number of moles of oxyethylene less than or equal to 10. The use of such an ester of sorbitan in the emulsifying system can lower the potential for irritation of the eyes relative to a conventional shampoo containing only detergent surfactants.

European Patent No. 1 430 867 describes an improved method for the preparation of cationic nanoemulsions of the oil-in-water type, intended for the cosmetic treatment of keratinous materials. These nanoemulsions comprise at least one cationic surfactant, and at least one nonionic surfactant. The nonionic surfactants that may be used for the preparation of these nanoemulsions include, among others, the mono-oleates of sorbitan with 4, with 5 or with 20 OE.

International Patent Application No. 2004/089318 describes, for instance, compositions of shampoos offering improved detergent and anti-dandruff action. These compositions contain:

(a) from 5 to 50 wt. % of a detergent surfactant,

(b) from 0.1 to 4 wt. % of an anti-dandruff agent,

(c) from 0.1 to 50 wt. % of a polyoxyethylenated derivative of an ester of fatty acid and sorbitan, and

(d) at least 20 wt. % of water.

Compound (c) is derived from an ester of sorbitan and a C₁₄-C₁₈ fatty acid such as palmitic, stearic, or oleic acid. It is used as a surfactant.

European Patent Application No. 0 490 053 describes a hair care composition in the form of a microemulsion comprising:

(a) from 5 to 20 wt. % of a nonionic surfactant with HLB ranging from 5 to 12 or of a mixture of such surfactants, the HLB of the mixture ranging from 6 to 10,

(b) from 5 to 20 wt. % of at least one oil,

(c) from 0.5 to 10 wt. % of at least one cationic surfactant, and

(d) from 50 to 89.5 wt. % of water,

this composition not comprising a nonionic surfactant or mixture thereof with of HLB greater than 12. Among the nonionic surfactants that can be used in these microemulsions, non-limiting mention may be made of the oxyethylenated esters of C₁₂-C₁₈ fatty acids and of sorbitan comprising from 1 to 6 OE.

Thus, oxyalkylenated sorbitan esters have been used, in the hairdressing compositions described in the prior art, as surfactants or emulsifiers.

The present inventors have now discovered, surprisingly, that the use, in an aqueous or aqueous-alcoholic medium, of certain oxyalkylenated esters of fatty acids and sorbitan provides very effective soothing of the scalp, i.e., prevents the appearance of reactions of discomfort, lessens the intensity of these reactions, or even causes them to disappear.

In at least one embodiment, these esters can be used for preparing lotions or other hair care products that are effective against reactions of discomfort of the scalp, such as irritation, itching, tingling, and erythema.

The present disclosure therefore relates, in at least one embodiment, to a cosmetic method for soothing the reactions of discomfort of the scalp comprising applying on the scalp a cosmetic composition comprising, in an aqueous or aqueous-alcoholic medium, at least one ester of fatty acid and sorbitan comprising from 1 to 20 oxyalkylene units as a soothing agent.

Additionally, the present disclosure also relates to a method of preparation of an aqueous or aqueous-alcoholic cosmetic composition intended for soothing the reactions of discomfort of the scalp, comprising incorporating in said composition at least one ester of fatty acid and sorbitan comprising from 1 to 20 oxyalkylene units as a soothing agent.

The present disclosure further relates to a method of cosmetic treatment of the reactions of discomfort of the scalp, comprising applying on the scalp a cosmetic composition comprising, in an aqueous or aqueous-alcoholic medium, a soothing agent comprising at least one ester of fatty acid and sorbitan comprising from 1 to 20 oxyalkylene units.

As used in the present disclosure, the phrase “soothing the reactions of discomfort of the scalp” is understood to mean preventing the development of at least one reaction of discomfort of the scalp, such as hotness, itching, tingling, erythema, irritation, as well as reducing the intensity of such a reaction, or even eliminating it, when the latter is already present on the scalp.

Other objects and characteristics, aspects and advantages of the disclosure will become clearer on reading the description and the examples given below.

As used herein, “compound with X OE” is understood to mean an oxyethylenated compound comprising X oxyethylene units per molecule.

Furthermore, as used in the present disclosure, “CY compound” is understood to mean a compound comprising Y carbon atoms.

Non-limiting examples of the compounds that can be used in the present disclosure are esters of fatty acid and sorbitan comprising from 1 to 20 oxyalkylene units. The oxyalkylene units can, for example, be chosen from oxyethylene and oxypropylene units, and, in at least one embodiment, are oxyethylene units.

In at least one embodiment, oxyethylenated derivatives of mono and polyesters of C₈₋₃₀ fatty acids and sorbitan having from 1 to 20 ethylene oxide units are used. In a further embodiment, oxyethylenated derivatives of mono and polyesters of C₁₂₋₂₄ fatty acids and sorbitan having from 4 to 20 ethylene oxide units are used.

Such compounds are also called polysorbates. Among others, they are marketed under the designation TWEEN by the company UNIQEMA. Non-limiting examples can be made of the following: the oxyethylenated sorbitan monolaurate with 4 OE, marketed under the designation TWEEN 21, the oxyethylenated sorbitan monolaurate with 20 OE, marketed under the designation TWEEN 20, the oxyethylenated sorbitan monopalmitate with 20 OE marketed under the designation TWEEN 40, the oxyethylenated sorbitan monostearate with 20 OE marketed under the designation TWEEN 60, the oxyethylenated sorbitan monostearate with 4 OE marketed under the designation TWEEN 61, the oxyethylenated sorbitan tristearate with 20 OE marketed under the designation TWEEN 65, the oxyethylenated sorbitan monooleate with 20 OE marketed under the designation TWEEN 80, the oxyethylenated sorbitan monooleate with 5 OE marketed under the designation TWEEN 81, and the oxyethylenated sorbitan trioleate with 20 OE marketed under the designation TWEEN 85.

The fatty acid of the oxyalkylenated sorbitan ester can be, for example, a saturated fatty acid.

In at least one embodiment, the sorbitan esters used herein are the oxyethylenated sorbitan monolaurate with 4 OE, the oxyethylenated sorbitan monolaurate with 20 OE, and mixtures thereof. In at least one further embodiment, the sorbitan ester is the oxyethylenated sorbitan monolaurate with 4 OE.

According to the present disclosure, the oxyalkylenated ester of fatty acid and sorbitan can be employed in an aqueous medium, or in an aqueous-alcoholic medium.

As used in the present disclosure, the term “aqueous medium” is understood to mean a medium comprising at least 50 wt. % of water.

As used in the present disclosure, the term “aqueous-alcoholic medium” is understood to mean a medium comprising a mixture of water and at least one cosmetically acceptable alcohol and/or at least one cosmetically acceptable polyol.

The aqueous-alcoholic medium can thus comprise at least one polyol chosen from glycerol, propylene glycol, polyethylene glycols and mixtures thereof.

In at least one embodiment of the present disclosure, the aqueous-alcoholic medium comprises at least one C₁-C₄ lower alcohol, for example ethanol, isopropanol, tert-butanol, n-butanol and mixtures thereof. In at least one further embodiment, the aqueous-alcoholic medium comprises ethanol.

It is also possible to use, within the scope of the present disclosure, a mixture of alcohol(s) and of polyol(s) as described above.

The weight ratio of water to alcohol(s) and polyol(s), in the aqueous-alcoholic medium, can, for example, range from 0.05 to 20. For instance, this ratio can range from 0.1 to 10, such as from 0.25 to 4, and further, for example, from 0.5 to 2.

The total content of alcohol(s) and polyol(s) in the aqueous-alcoholic medium can range from 5 to 95 wt. %, for example from 10 to 90 wt. %, further for example from 20 to 80 wt. %, and even further for example from 20 to 70 wt. %.

According to the present disclosure, the oxyalkylenated ester of fatty acid and sorbitan can be applied to the scalp in an aqueous composition, i.e., a composition comprising at least 50 wt. % of water, or in an aqueous-alcoholic composition, i.e., a composition comprising at least 50 wt. % of the aforementioned mixture of water and of alcohol(s) and/or polyol(s).

For this purpose, the oxyalkylenated ester of fatty acid and sorbitan can be incorporated as a soothing agent in any hair treatment composition that comprises water and/or at least one alcohol and/or at least one polyol.

For example, the oxyalkylenated ester of the fatty acid and sorbitan can thus be added as a soothing agent to conventional hair care compositions, such as compositions for washing the hair and the scalp, compositions for coloring, bleaching, perming or straightening of the hair, compositions for shaping the hair such as lacquers, gels, mousses, compositions for cosmetic treatment of the scalp such as anti-dandruff treatments, treatments against hair loss, moisturizing treatments, nourishing treatments, and soothing treatments.

Thus, the oxyalkylenated ester of fatty acid and sorbitan can be added, for example, to a shampoo or after-shampoo composition.

Shampoo and after-shampoo compositions are familiar to a person skilled in the art. Shampoos conventionally comprise a washing base comprising at least one surfactant chosen from anionic, nonionic, amphoteric and zwitterionic surfactants. The after-shampoos conventionally comprise at least one conditioner, for example cationic surfactants, cationic polymers, amphoteric or zwitterionic polymers, or silicones. The shampoos can also comprise such conditioners.

The shampoos and after-shampoos can also comprise, non-exhaustively, at least one fat, for example vegetable oils, animal oils, mineral oils, natural or synthetic oils, waxes, and aliphatic alcohols, at least one conventional additive such as agents against hair loss, oxidants, ceramides and pseudo-ceramides, vitamins and pro-vitamins including panthenol, ceramides and pseudo-ceramides, sun filters, mineral or organic, colored or colorless pigments, dyes, lustre agents and opacifiers, sequestering agents, plasticizers, solubilizers, acidifiers, alkalizing agents, mineral or organic thickeners, antioxidants, hydroxy acids, perfumes and preservatives.

The oxyalkylenated ester of fatty acid and sorbitan can also be added to anti-dandruff compositions such as anti-dandruff shampoos, and anti-dandruff after-shampoos.

The anti-dandruff shampoos and after-shampoos comprise, in addition to the conventional ingredients of shampoos and after-shampoos, at least one anti-dandruff agent. The following are non-limiting examples of compounds that can be used as anti-dandruff agents: piroctone olamine, zinc pyrithione, salicylic acid, selenium disulphide, and mixtures thereof.

The compositions of anti-dandruff shampoos and after-shampoos comprise, in at least one embodiment, from 0.001 to 10 wt. % of anti-dandruff agent(s), for example, from 0.1 to 5 wt. %, further, for example, from 0.2 to 2 wt. %, relative to the total weight of the composition.

According to another embodiment of the present disclosure, the oxyalkylenated ester of fatty acid and sorbitan is incorporated in an aqueous-alcoholic lotion for cosmetic treatment of the scalp.

As used herein, the term “aqueous-alcoholic lotion” is understood to mean a lotion comprising at least 80 wt. %, for example at least 90 wt. %, and further for example at least 95 wt. % of a mixture of water and at least one alcohol and/or polyol as described above. It can be a conventional lotion, or an anti-dandruff lotion, i.e., a lotion comprising at least one anti-dandruff agent such as those described above.

Such an anti-dandruff lotion comprises, in at least one embodiment, from 0.001 to 10 wt. % of anti-dandruff agent(s), for example from 0.01 to 5 wt. %, and further for example from 0.02 to 2 wt. %, relative to the total weight of the composition.

According to at least one embodiment of the present disclosure, the composition applied to the scalp comprises at least 0.01 wt. % of oxyalkylenated ester of fatty acid and sorbitan, relative to the total weight of the composition. For instance, the composition can comprise from 0.05 wt. % to 10 wt. % of oxyalkylenated ester of fatty acid and sorbitan, further for example from 0.1 to 8 wt. %, and even further for example, from 0.2 to 5 wt. %, relative to the total weight of the composition.

The present disclosure also relates to a method of cosmetic treatment intended for soothing reactions of discomfort of the scalp, comprising applying, on the scalp, an effective amount of a cosmetic composition as described above and which comprises, in an aqueous or aqueous-alcoholic medium, as a soothing agent, at least one oxyalkylenated ester of fatty acid and sorbitan as described herein.

According to at least one embodiment of the present disclosure, such a method comprises applying, on the scalp, an effective amount of the cosmetic composition, then optionally rinsing, optionally after a waiting time.

The method according to the present disclosure can be employed with or without rinsing. In at least one embodiment, it is employed without rinsing, i.e., it does not include a rinsing stage.

In yet another embodiment of the present disclosure, the method comprises applying an effective amount of an aqueous-alcoholic lotion comprising at least one oxyalkylenated ester of fatty acid and sorbitan as described above, to the scalp when dry or wet. The product is then optionally rinsed, after a waiting time ranging from 30 s to 10 min.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the present disclosure in a non-limiting manner.

### EXAMPLE

In the following comparative example, all the amounts are given as percentage by weight of active substance, relative to the total weight of the composition, unless stated otherwise.

A lotion A, according to the present disclosure, and a comparative lotion B were prepared from the ingredients shown in the following table.

| Composition | Lotion A | Lotion B |
|---|---|---|
| oxyethylenated sorbitan monolaurate | 0.5 | 0 |
| with 4 OE (TWEEN 21 from Uniqema) | | |
| ethoxylated castor oil (CREMOPHOR | 0.3 | 0.3 |
| RH 40 from BASF) | | |
| ethanol | 40.6 | 40.6 |
| water qsf | 100% | 100% |

A test of efficacy of the soothing effect of lotions A and B with respect to an irritant (capsaicin) was carried out, as follows:

Lotions A and B were each applied on a small area of the scalp (the hair having been shaved from the areas of application) of 29 subjects with sensitive scalps.

Then a cream comprising 0.025 wt. % of capsaicin was applied to the two application areas of each subject.

The reactions of discomfort of the scalp (hotness, itching, tingling and erythema) were evaluated before each application of product on the scalp, then 10 minutes after the waiting time for each lotion, then at different waiting times for the cream based on capsaicin.

The sensations of hotness, itching and tingling were each assessed and noted by each subject, on an increasing scale (with the score increasing as the sensation felt increases). Development of erythema was observed and noted (also on an increasing scale), for each subject, by the experimenter.

The reactions of discomfort obtained with lotion A on one hand, and with lotion B on the other hand, were analysed statistically (mean value for each reaction of discomfort, and for the cumulative discomfort: hotness+itching+tingling+erythema).

The following results were obtained:

regarding the reactions of erythema, the mean value obtained was 4.3 for lotion A, and 6.7 for lotion B, with a value of p of 0.005.

regarding the cumulative discomfort, the mean value obtained was 10 for lotion A, and 14.1 for lotion B, with a value of p of 0.020.

Thus, application of lotion A according to the present disclosure on the scalp was found to provide a soothing effect far greater than that obtained by application of comparative lotion B.

In particular, a significant decrease in reactions of erythema, as well as cumulative discomfort, was observed.

What is claimed is:

1. A cosmetic method for soothing reactions of discomfort of the scalp, comprising applying on the scalp as a soothing agent a cosmetic composition comprising, in an aqueous or aqueous-alcoholic medium, at least one ester of fatty acid and sorbitan comprising from 1 to 20 oxyalkylene units.

2. A method according to claim 1, wherein the at least one oxyalkylenated ester of fatty acid and sorbitan is oxyethylenated.

3. A method according to claim 2, wherein the at least one ester of fatty acid and sorbitan is chosen from oxyethylenated derivatives of mono and polyesters of C₈₋₃₀ fatty acids and sorbitan, comprising from 1 to 20 ethylene oxide units.

4. A method according to claim 3, wherein the at least one ester of fatty acid and sorbitan is chosen from oxyethylenated derivatives of mono and polyesters of C₁₂₋₂₄ fatty acids and sorbitan, comprising from 4 to 20 ethylene oxide units.

5. A method according to claim 4, wherein the at least one ester of fatty acid and sorbitan is chosen from oxyethylenated sorbitan monolaurate with 4 OE, oxyethylenated sorbitan monolaurate with 20 OE, and mixtures thereof.

6. A method according to claim 5, wherein the at least one ester of fatty acid and sorbitan is oxyethylenated sorbitan monolaurate with 4 OE.

7. A method according to claim 1, wherein the composition comprises an aqueous medium.

8. A method according to claim 1, wherein the composition comprises an aqueous-alcoholic medium comprising a mixture of water and at least one cosmetically acceptable alcohol and/or at least one cosmetically acceptable polyol.

9. A method according to claim 8, wherein the composition comprises an aqueous-alcoholic medium comprising at least one polyol chosen from glycerol, propylene glycol, polyethylene glycols and mixtures thereof.

10. A method according to claim 8, wherein the composition comprises an aqueous-alcoholic medium comprising at least one lower C₁-C₄ alcohol.

11. A method according to claim 10, wherein the at least one lower C₁-C₄ alcohol is chosen from ethanol, isopropanol, tert-butanol, n-butanol and mixtures thereof.

12. A method according to claim 11, wherein the aqueous-alcoholic medium comprises ethanol.

13. A method according to claim 8, wherein, in the aqueous-alcoholic medium, the weight ratio of the water to the alcohol(s) and polyol(s) ranges from 0.05 to 20.

14. A method according to claim 13, wherein the weight ratio of water to alcohol(s) and polyol(s) ranges from 0.1 to 10.

15. A method according to claim 8, wherein the total content of alcohol(s) and polyol(s) in the aqueous-alcoholic medium ranges from 5 to 95 wt. %.

16. A method according to claim 15 wherein the total content of alcohol(s) and polyol(s) in the aqueous alcoholic medium ranges from 20 to 80 wt. %.

17. A method according to claim 1, wherein the cosmetic composition is a shampoo or after-shampoo composition comprising, as a soothing agent, said at least one ester of fatty acid and sorbitan

18. A method according to claim 17, wherein the shampoo or after-shampoo composition is an anti-dandruff shampoo or an anti-dandruff after-shampoo comprising, in addition to the conventional ingredients of shampoos and after-shampoos, at least one anti-dandruff agent.

19. A method according to claim 1, wherein the cosmetic composition is an aqueous-alcholic lotion for treatment of the scalp, comprising, as a soothing agent, said at least one ester of fatty acid and sorbitan.

20. A method according to claim 19, wherein the aqueous-alcoholic lotion comprises at least 80 wt. % of a mixture of water and alcohol(s) and/or polyol(s).

21. A method according to claim 20, wherein the aqueous-alcoholic lotion comprises at least 95 wt. % of a mixture of water and alcohol(s) and/or polyol(s).

22. A method according to claim 1, wherein the cosmetic composition is an anti-dandruff lotion comprising at least one anti-dandruff agent and, as a soothing agent, at least one ester of fatty acid and sorbitan.

23. A method according to claim 22, wherein the anti-dandruff agent is chosen from piroctone olamine, zinc pyrithione, salicylic acid, selenium disulphide and mixtures thereof.

24. A method according to claim 18, wherein the anti-dandruff shampoo or the anti-dandruff after-shampoo comprises from 0.001 to 10 wt. % of anti-dandruff agent(s), relative to the total weight of the composition.

25. A method according to claim 22, wherein the anti-dandruff lotion comprises from 0.001 to 10 wt. % of anti-dandruff agent(s), relative to the total weight of the composition.

26. A method according to claim 1, wherein the composition applied to the scalp comprises at least 0.01 wt. % of the at least one ester of fatty acid and sorbitan, relative to the total weight of the composition.

27. A method according to claim 26, wherein the composition applied to the scalp comprises from 0.05% to 10 wt. % of the at least one ester of fatty acid and sorbitan relative to the total weight of the composition.

28. A method according to claim 27, wherein the composition applied to the scalp comprises from 0.2% to 5 wt. % of the at least one ester of fatty acid and sorbitan relative to the total weight of the composition.

29. A cosmetic method for soothing reactions of discomfort of the scalp, comprising
applying on the scalp an effective amount for soothing of a cosmetic composition comprising, in an aqueous or aqueous-alcoholic medium, at least one ester of fatty acid and sorbitan comprising from 1 to 20 oxyalkylene units, and
optionally rinsing the scalp, optionally after a waiting time.

30. A method according to claim 29, which does not include a rinsing stage.

31. A cosmetic method for soothing reactions of discomfort of the scalp, comprising
applying, on a dry or wet scalp, an effective amount for soothing of a cosmetic composition comprising an aqueous-alcoholic lotion comprising at least one oxyalkylenated ester of fatty acid and sorbitan, and
optionally rinsing the scalp after a waiting time ranging from 30 s to 10 min.
